(19) United States
(12) Patent Application Publication
(10) Pub. No.: US 2009/0292307 A1
(43) Pub. Date: Nov. 26, 2009

(54) MECHANICAL EMBOLECTOMY DEVICE AND METHOD

(76) Inventor: NASSER RAZACK, Bradenton, FL (US)

Correspondence Address: Maxey Law Offices, PLLC, 13630 58TH ST. NORTH, SUITE 101, CLEARWATER, FL, 33760, US

(21) Appl. No.: 12/125,591

(22) Filed: May 22, 2008

(51) Int. Cl. A61M 29/00

(52) U.S. Cl. 606/200; 606/191

(57) ABSTRACT

An embolectomy device includes a catheter and an elongated shaft positioned in and moveable within the catheter. The shaft has proximal and distal end portions, and a retrieval portion between the proximal and distal end portions. The retrieval portion has a plurality of legs having proximal and distal end portions. Portions of the legs are movable laterally outward from a long axis of the elongated shaft when the retrieval portion is at least partially moved out of the catheter. A retrieval net can be fixed to portions of the legs for engaging a clot or other occlusion within a body canal. An expander can be provided for moving portions of an occlusion toward canal walls to open a lumen. A method for removing clots and other occlusions from body canals is also disclosed.

### BACKGROUND OF THE INVENTION

There are currently in excess of 700,000 new or recurrent strokes every year in the United States. It is the third leading cause of death in the United States after coronary artery disease and cancer. Approximately 40% (>250,000) of strokes are due to large vessel occlusion, potentially requiring a device for treatment.

Treatment must begin with an evaluation of the patient. Diagnostic neuroimaging is used to obtain noninvasive real-time information about the patient. The goal of therapy is to restore perfusion to the ischemic but potentially salvageable brain tissue rather than to the irreversibly damaged brain tissue, since re-establishing blood flow to such damaged tissue can cause complications such as hemorrhage. Determining the cause and location of the blockage is critical to planning the treatment approach. The most common cause of ischemic stroke is acute embolic occlusion. Most patients with acute ischemic stroke have thromboembolic material occluding large cerebral vessels and hence disruption of cerebral blood flow. Removal of the arterial occlusion in a timely manner can provide a substantial reduction in the size and severity of the cerebral infarction, and improvement in the level of disability among survivors. Treatment varies depending on whether the lesion is proximal or distal, whether there is underlying atherosclerotic stenosis at the occlusion site, and whether the proximal extracranial vessel is opened or closed. Where there are proximal arterial occlusions, the physician may attempt clot retrieval, supplemented by direct catheter-directed thrombolysis. Severe stenosis proximal to the occlusion will usually require treatment of the stenosois before or immediately after restoring intracranial flow.

A current treatment for acute ischemic stroke is intravenous thrombolysis using tissue-type plasminogen activator (TPA). TPA is a naturally occurring enzyme that activates plasminogen into active plasmin, which dissolves fibrin. The dissolution of fibrin in a clot causes thrombolysis. This treatment is suitable for smaller clots, but has limited utility for patients with large clots, such as are often present in acute occlusions of the internal carotid artery (ICA), proximal middle cerebral artery (MCA), and basilar artery (BA). Also, TPA therapy has significant time constraints, and is generally effective only if given within 3-6 hours of stroke symptom onset. Contraindications to TPA and these time constraints led to mechanical embolectomy.

Mechanical removal of the thrombus is the goal of mechanical embolectomy. Lytic therapy is necessary for non-accessible locations. Mechanical embolectomy is the process by which a mechanical device is inserted into the body, moved through the affected body canal to the site of the occlusion, and then used to mechanically remove the occlusion from the canal to restore blood flow. One such device is the Mechanical Embolus Removal in Cerebral Ischemia (Merci) retrieval device (Concentric Medical, Mountain View, Calif.), which is currently available for routine clinical use in acute ischemic stroke within 9 hours of onset. This device is a flexible and tapered nickel titanium wire with a helically shaped distal tip that can be deployed intra-arterially to entrap and retrieve large vessel intracerebral clots. Other devices are in various stages of development.

### SUMMARY OF THE INVENTION

An embolectomy device includes a catheter and an elongated shaft positioned in and moveable within the catheter. The shaft has proximal and distal end portions, and a retrieval portion at or near the distal end portion. The retrieval portion has a plurality of legs having proximal and distal ends and a mid-portion between the proximal and distal ends. The mid-portion of the legs is movable laterally outward from a long axis of the elongated shaft when the retrieval portion has been moved out of the catheter. A retrieval net can be fixed to the legs between the mid-portion and the distal end of the legs.

The elongated shaft can be a guide wire. The legs can be spring arms. The spring arms can comprise a bend at the mid portion defining proximal and distal portions, and the bend can bias the spring arms to a lateral outward position. The net can be constructed from a polymeric material. The legs can be integral with the elongated shaft, or can be affixed to the elongated shaft. The elongated shaft can comprise a distal penetrating portion.

An embolectomy device includes a catheter and an elongated shaft positioned in and moveable within the catheter. The shaft has proximal and distal end portions, and a retrieval portion at or near the distal end portion. The retrieval portion has a plurality of legs having proximal and distal portions. A portion of the legs are movable laterally outward from a long axis of the elongated shaft when the retrieval portion has been at least partially moved out of the catheter. A retrieval net is fixed to the distal portions of the plurality of legs.

A method of performing an embolectomy includes the step of providing an embolectomy device comprising a catheter; an elongated shaft positioned in and moveable within the catheter; the shaft having proximal and distal end portions; the shaft having a retrieval portion at or near the distal end portion; the retrieval portion having a plurality of legs having proximal and distal end portions; at least a portion of the legs being movable laterally outward from a long axis of the elongated shaft when the retrieval portion has been moved at least partially out of the catheter; and a retrieval net fixed to the distal portions of the plurality of legs. The embolectomy device is manipulated to a location adjacent to an occlusion in a body canal. At least the retrieval portion is moved through the occlusion. The retrieval portion is moved at least partially out of the catheter, and at least a portion of the legs are moved laterally outward. The retrieval portion is moved proximally so as to engage the occlusion with the retrieval net. The microcatheter, elongated shaft, retrieval portion, and occlusion are then removed from the body canal.

### BRIEF DESCRIPTION OF THE DRAWINGS

There is shown in the drawings embodiments which are presently preferred, it being understood, however, that the invention can be embodied in other forms without departing from the spirit or essential attributes thereof.

FIG. 1 is a side elevation of an embolectomy device according to the invention in a first mode of operation.

FIG. 2 is a side elevation of an embolectomy device according to the invention in a second mode of operation.

FIG. 3 is a side elevation of an embolectomy device according to the invention in a third mode of operation.

FIG. 4 is a side elevation of an embolectomy device according to the invention in a body canal and in a first mode of operation.

FIG. 5 is a side elevation of an embolectomy device according to the invention in a body canal and in a second mode of operation.

FIG. 6 is a side elevation of an embolectomy device according to the invention in a body canal and in a third mode of operation.

FIG. 7 is a side elevation of an alternative embodiment.

FIG. 8 is a side elevation of another alternative embodiment.

FIG. 9(A-B) is a perspective view of a microcatheter.

FIG. 10(A-B) is a perspective view of an alternative embodiment.

FIG. 11(A) is a side elevation of a retrieval portion in a vibration mode of operation.

FIG. 11(B) is a side elevation of a retrieval portion in a spinning mode of operation.

FIG. 12 is a cross section illustrating an embolectomy device with an expander, in a first mode of operation.

FIG. 13 is a cross section illustrating an embolectomy device with an expander, in a second mode of operation.

FIG. 14 is a cross section illustrating an embolectomy device with an expander, in a third mode of operation.

FIG. 15 is a cross section illustrating an embolectomy device with an expander, in a fourth mode of operation.

FIG. 16 is a cross section of an embolectomy device with an expander and a retrieval portion, in a first mode of operation.

FIG. 17 is a cross section of an embolectomy device with an expander and a retrieval portion, in a second mode of operation.

### DETAILED DESCRIPTION OF THE INVENTION

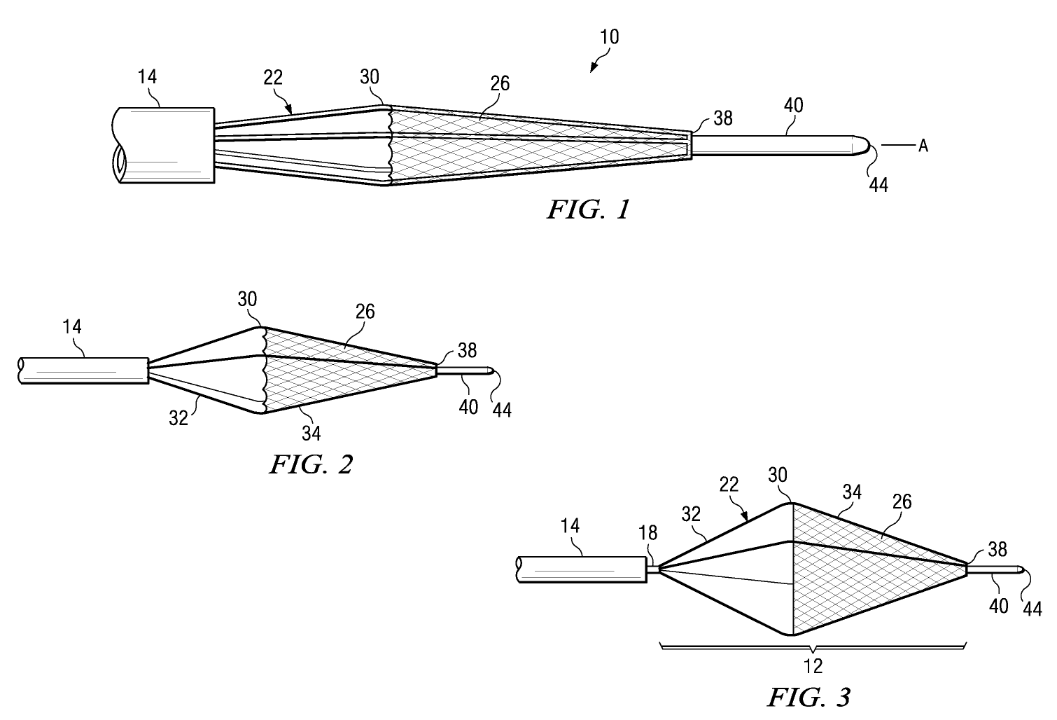

There is shown in FIGS. 1-3 an embolectomy device 10 according to the invention. The embolectomy device 10 comprises a microcatheter 14 having an elongated shaft such as guide wire 18 extending through an opening in the microcatheter 14. The elongated shaft 18 can have proximal and distal end portions. A retrieval portion 12 can be provided at or near the distal end portion of the elongated shaft 18. The retrieval portion may be formed from or attached to the elongated shaft 18. The retrieval portion 12 can have a plurality of legs such as spring arms 22 that are connected to the guide wire 18. The spring arms 22 are disposed about a long axis A of the guide wire 18. The spring arms 22 are biased or otherwise moveable to extend partially outwardly (FIG. 2) when not constrained entirely by the microcatheter 14. When unconstrained by the microcatheter 14 (FIG. 3) the spring arms 22 can extend laterally outward a maximum distance. Movement of the spring arms 22 relative to the microcatheter 14 can thereby be used to control the lateral extension of the spring arms 22 within a range of possible distances between full extension and full retraction.

A retrieval net 26 can be provided on the spring arms 22 to assist in engaging and removing a clot or other obstruction from a body canal. The net 26 can be constructed of a flexible, thin-walled material such that when the spring arms 22 are positioned within the microcatheter 14, the net can also be contained within the microcatheter 14. The net 26 can also move freely into and out of the microcatheter 14 with the retrieval portion 12. The net 26 can be constructed from a porous or a substantially non-porous material, such as a flexible plastic net or solid sheet material, and/or a biocompatible or non-thrombogenic polymer. The net 26 can be formed from a polymeric material that is adhered or otherwise securely fixed to the spring arms 22.

The precise arrangement and construction of the spring arms 22 can be varied. In the embodiment shown, the spring arms 22 are elongated and substantially radially disposed about an axis A of the guide wire 18. The spring arms 22 can be formed separately and attached to the guide wire 18, or the spring arms 22 can be integral with the guide wire 18 and formed by suitable techniques such as computer-controlled laser cutting. The spring arms can have a mid-portion 30, which can have a bend, crimp, curve, or other biasing or moving feature or structure which causes the legs/spring arms 22 to extend laterally outward relative to the long axis A of the guide wire 18. In one embodiment, the spring arms 22 are at least partially made of an elastic material, such as plastic or metal. The mid-portion 30 defines proximal portions 32 and distal portions 34 of the spring arms 22. When the proximal portions 32 are positioned within the microcatheter 14, the proximal portions 32 are retained in a laterally inward position by the inside wall of the microcatheter (FIG. 1). As the guide wire 18 is extended from the microcatheter 14, the spring arms 22 are permitted under the influence of the biasing to extend laterally outward (FIG. 2). As the guide wire 18 is moved further out of the microcatheter 14, the spring arms 22 are moved by the biasing completely out of the microcatheter 14 and extend a maximum lateral distance from the long axis A of the guide wire 18 (FIG. 3). The distal portions 34 extend from a lateral maximum distance at the bend 30 to a lateral minimum distance at the distal end 38 of the spring arms 22. The lateral outward extension of the legs/spring arms 22 can thereby be controlled by the distance which the proximal portions 32 are moved out of the microcatheter 14.

The net 26 is joined to the distal portions 34 of the spring arms 22, such that when the spring arms 22 are laterally extended, the net 26 is opened to what in one embodiment is a substantially conical configuration and retained in that position by the spring arms 22, as shown in FIG. 3. In this position, the net 26 can engage, capture and retain a clot or other obstruction during an embolectomy. The net 26 also retains debris which otherwise might be left in the body canal. A distal penetrating portion 40 can be provided for penetrating the clot such that the spring arms 22 and net 26 can be moved through the clot. The penetrating portion 40 can be part of the guide wire 18 or can be a separate structure to which the spring arms 22 are affixed. A tip 44 or other piercing or penetrating structure can be provided at the distal end of the penetrating portion 40 to assist in penetrating the clot to provide for passage of the spring arms 22 and net 26 through the clot. The tip 44 can be blunted so as not to damage the body canal during use.

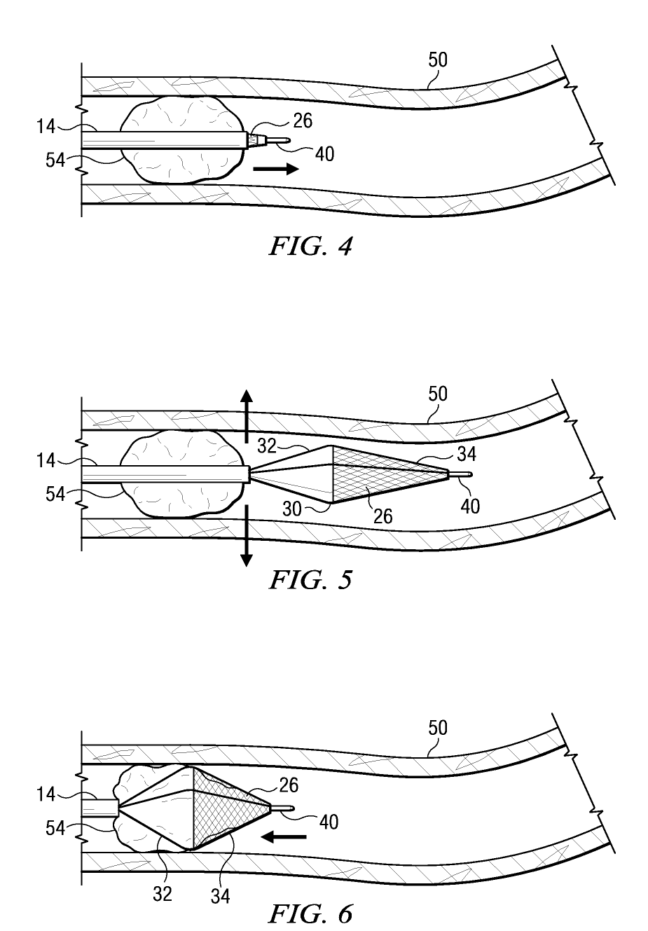

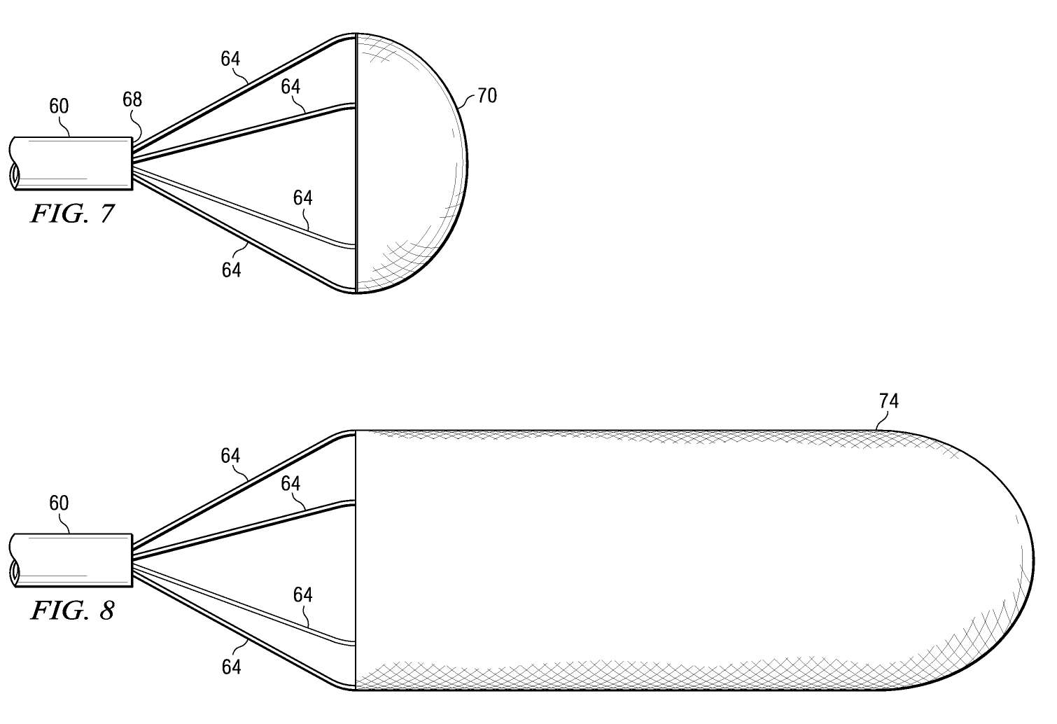

Operation of the embolectomy device is shown in FIGS. 4-6. The location of the clot is determined, and then the microcatheter 14 is moved into a position within a body canal 50 adjacent to the clot 54 by standard interventional radiology techniques. The guide wire 18, retrieval portion 12, and penetrating portion 40 can be completely retracted within the microcatheter 14 to avoid damage to the body canal 50 as the microcatheter 14 is moved through the body canal 50. The penetrating portion 40 and also possibly the microcatheter 14 are then moved through the clot 54 (FIG. 4). The guide wire 18 is extended from the microcatheter 14, either by withdrawing the microcatheter 14, or moving the guide wire 18 through the microcatheter 14, or some combination of both. As the proximal portions 32 emerge from the microcatheter 14 (FIG. 5), the spring arms 22 begin to extend laterally outward, which extends the mid-portions 30 laterally outward. If the spring arms 22 are moved completely out of the microcatheter 14 (FIG. 6), the spring arms 22 extend outward a maximum lateral distance relative to the long axis A of the guide wire 18. The positioning of the guide wire 18 and spring arms 22 relative to the microcatheter 14 can thereby be used to control the lateral distance which the spring arms 22 extend outward. This is significant because the dimensions of the inside diameter of a body canal can vary, and the lateral extension of the spring arms 22 can be controlled according to the inside diameter of the body canal. The guide wire 18 can then be retracted, and the clot 54 will be engaged by the net 26. As the microcatheter 14 and guide wire 18 are removed from canal 50, the net 26 will retain the clot 54 and remove the clot 54 from the canal 50.

The dimensions and construction of the microcatheter 14, elongated shaft or guide wire 18, and legs/spring arms 22 can vary depending on the size of the canal in which the clot is located, the size and position of the clot 54, and other factors. The dimensions of the retrieval portion 12 can, for example, be between 0.20 mm to 0.45 mm in diameter when collapsed, and between 0.4 mm to 10 mm when open. In one embodiment the retrieval portion 12 can have a length between 2 mm and 22 mm. The dimensions of the elongated shaft can in one embodiment be 0.35 mm in diameter, and between 0.20 mm to 0.45 mm in diameter. The dimensions of the penetrating portion can be 0.25 mm in diameter, or between 0.20 mm and 0.45 mm in diameter. The dimensions of the microcatheter 14 can be an outside diameter (OD) 0.60 mm, and an inside diameter (ID) of 0.43 mm, or with an OD between 0.40 mm to 1.37 mm, and an ID between 0.25 mm to 0.75 mm. Other dimensions are possible.

The instruments used to position and manipulate the microcatheter 14 and the guide wire 18 can be standard devices or devices specifically designed for use with the invention. Although the legs have been described as spring arms 22, the invention is also useful when the legs/spring arms 22 are moved laterally outward by a force other than a spring force, as where the legs are driven laterally outward by a motor of some kind, such as an osmotic pump. In other embodiments, the mid-portion 30 of the legs/spring arms 22 can be at least partially elastic or can comprise a hinge structure to permit bending of the legs/spring arms.

Other embodiments are possible. There is shown in FIG. 7 an embodiment with a microcatheter 60. Struts or legs 64 move laterally outward when extended from opening 68 of the microcatheter. A flexible retrieval net 70 is attached to distal ends of the struts 64, and unfurls when the struts 64 are opened. The struts 64 and retrieval net 70 can be pushed through a clot, and then the struts 64 are moved laterally outward and the retrieval net 70 is unfolded. The clot is then engaged by the net and retrieved by removal of the microcatheter 60, struts 64 and net 70 from the body canal. The embodiment shown in FIG. 8 also has microcatheter 60 and struts 64, however, the net 74 in this embodiment is elongated and is therefore capable of capturing and securely retrieving significant amounts of clot debris or other biological material.

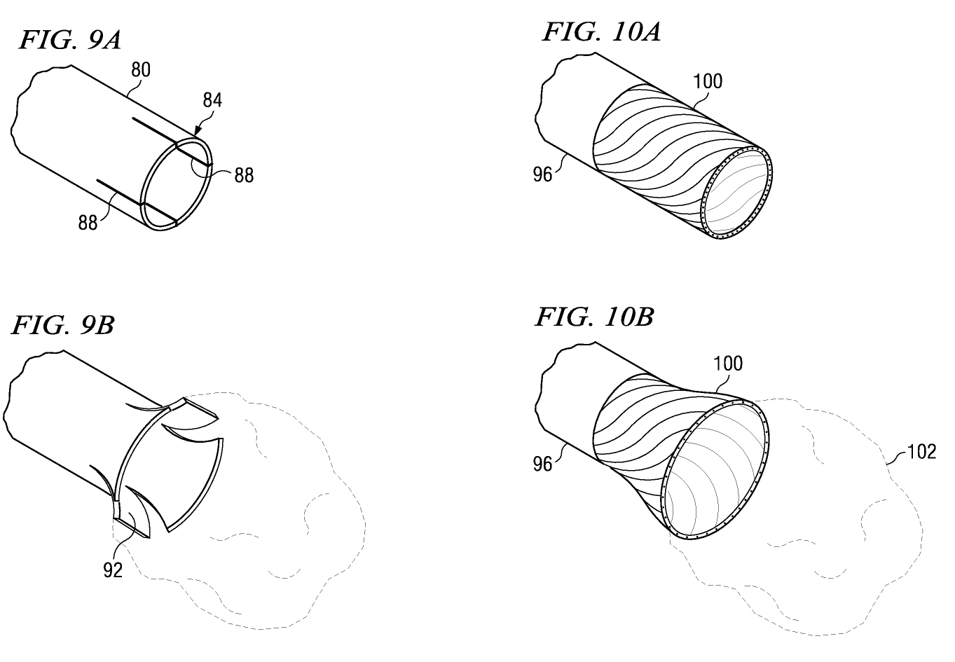

FIG. 9A depicts an alternative microcather 80 suitable for use with the invention in which the distal end 84 comprises an elastic or pliable material such as plastic or thin metal, such that the distal end 84 will expand upon receiving a clot to firmly retain the clot in the microcather 80 during the embolectomy process. Slots 88 can be provided at distal end 84 to create flanges 92 which bend more easily to permit the distal end 84 to expand and receive and retain the clot (FIG. 9B). In another embodiment shown in FIG. 10A-B, a microcatheter 96 is provided with an elastomeric tubular end material 100 (FIG. 10A) which expands or stretches to receive and engage a clot 102 (FIG. 10B).

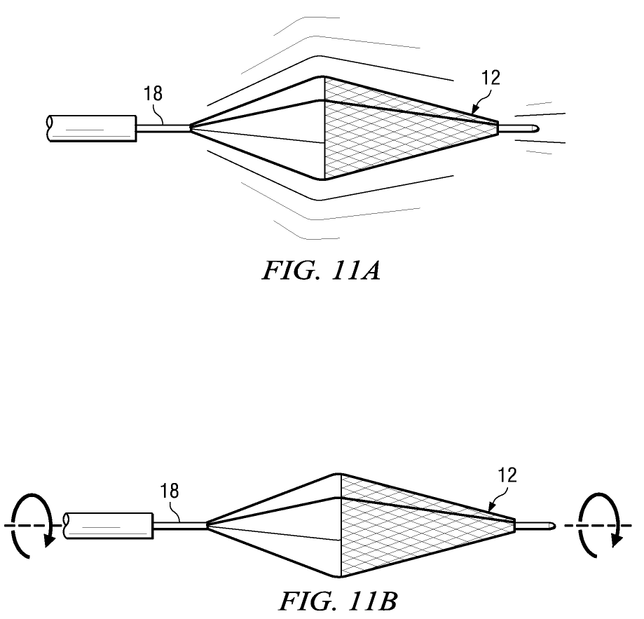

In FIG. 11(A-B) there is shown alternative methods by which the retrieval portion 12 can be moved to dislodge a clot. In FIG. 11A the retrieval portion 12 is vibrated, for example, by vibration of the guide wire 18 by suitable apparatus. In FIG. 11B the retrieval portion 12 is rotated, for example, by rotation of the guide wire 18. Other movements of the retrieval portion 12, such as a fast reciprocating motion, are also possible, as are other means for causing the movement of the retrieval portion 12.

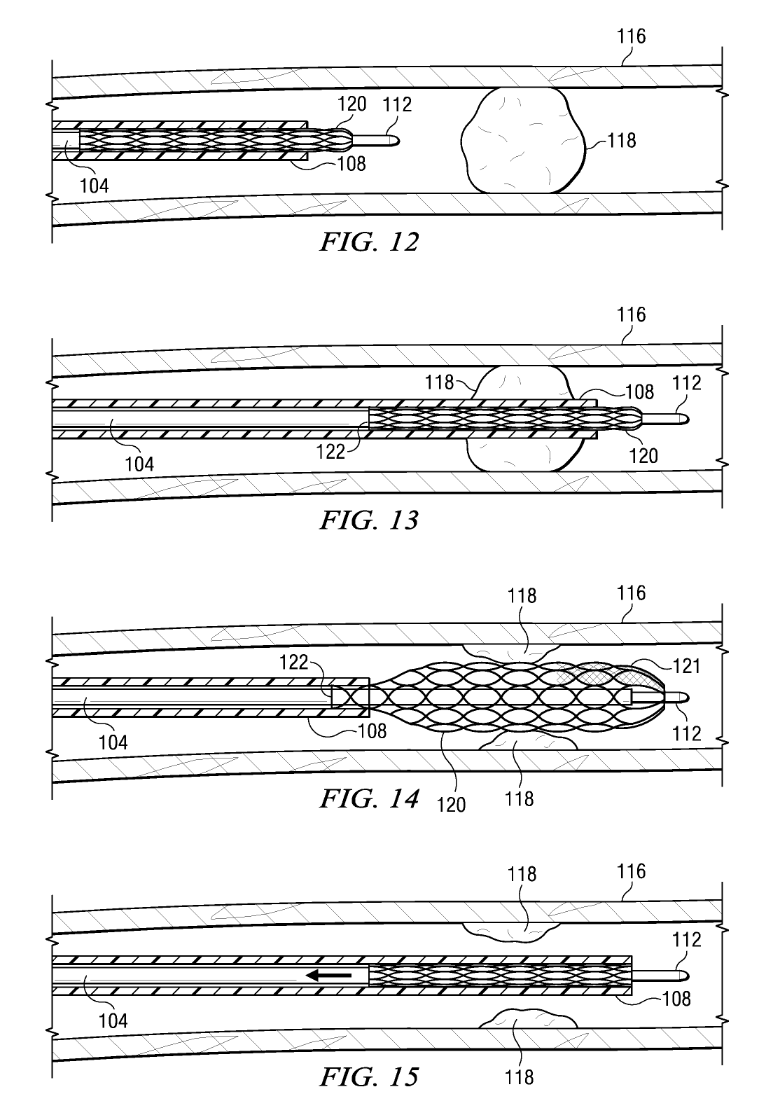

In FIGS. 12-15 there is shown the sequential operation of an alternative embodiment. In this embodiment, an inner catheter 104 is provided within an outer catheter 108. A guide wire 112 or other elongated shaft is provided within the inner catheter 104 and used to position the inner catheter 104 and outer catheter 108 within the body canal 116 (FIG. 12). The guide wire 112, inner catheter 104, and outer catheter 108 are then positioned through the clot of interest 118 (FIG. 13). The distal end of inner catheter 104 is provided with an expander 120, which is secured at a proximal end 122 to the inner catheter 104. The proximal end 122 can be partially contained within the outer catheter 108 in order to direct the expander 120 back into the outer catheter 108 when the device is to be withdrawn from the body canal. A mesh or net 121 can cover a portion or all of the expander 120 in order to better engage clot material. The net 121 can be constructed from a porous or a substantially non-porous material, such as a flexible plastic net or solid sheet material, and/or a biocompatible or non-thrombogenic polymer. Expansion of the expander 120 is controlled by suitable means, such as the presence of the outer catheter 108. When the outer catheter 108 is removed or pulled back from the expander 120, the expander 120 is expanded either by a spring force or a suitable motor, pressing the clot 118 against the body canal 116 (FIG. 14). The expander 120 can then be retracted into the outer catheter 108 (FIG. 15), whereupon the outer catheter 108, inner catheter 104 and guide wire 112 can be removed from the body canal 116, leaving a lumen for restoring blood flow. The outer catheter 108 can be of several different sizes and designs. The outer catheter 108 can have an inside diameter of 0.5 mm to 1.04 mm and an outside diameter of between 0.75 mm to 1.37 mm.

The expander 120 can be of different designs, but can be a mesh metallic or polymeric device similar to a stent in that it is capable of a first position in which it is tightly compacted laterally, and in another position expands laterally. The expander 120 uses this lateral expansion force to spread the clot material concentrically outwardly toward the walls of the body canal, opening a lumen through the body canal. In the high energy, compacted state the expander 120 is contained within a catheter. When moved out of the catheter, the expander 120 assumes an expanded, lower energy state. The expander 120 can be completely or partially coated with a polymer, and/or with a therapeutic substance. The expander 120 can be porous, non-porous, or partially porous.

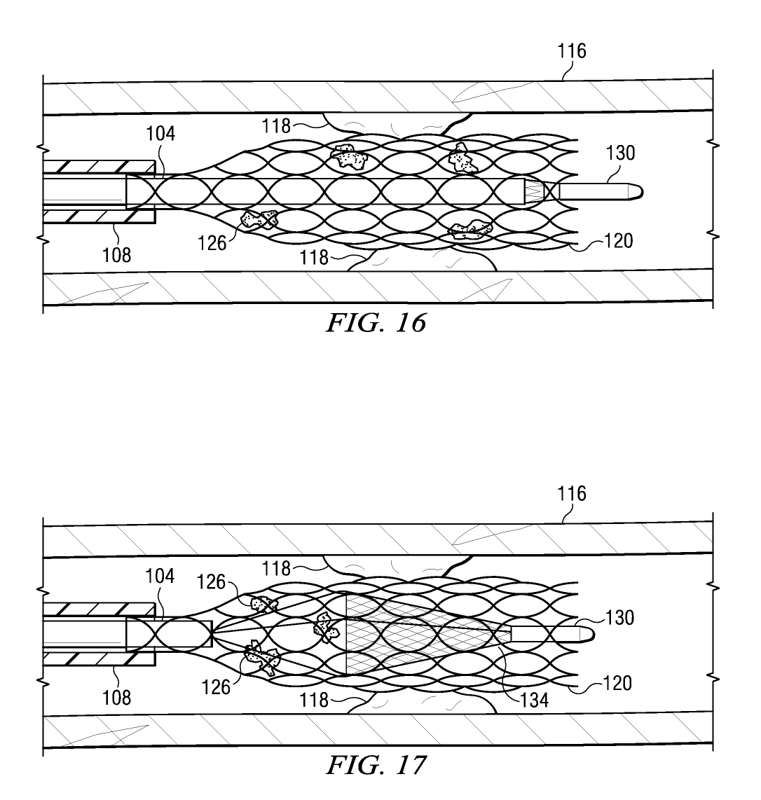

In FIGS. 16-17 there is shown an alternative embodiment to the device illustrated in FIGS. 12-15. The operation of the device is as described for FIGS. 12-15, however, in some instances remnants 126 of the clot 118 were not engaged by the expander 120 and pressed against body canal 116, and therefore remain within the expander 120 (FIG. 16). The guide wire 112 is then removed and replaced by a wire 130 having a retrieval portion 134. Extension of the wire 130 out of the inner catheter 104 will cause expansion of the retrieval portion 134, which can then be used to engage and remove clot remnants 126 (FIG. 17). Rather than replacing guide wire 112 with wire 130, it is also possible to provide the retrieval portion 134 on the guide wire 112 so that removal and replacement of the wires is not necessary. The retrieval portion can alternatively be other types of retrieval devices, such as the Merci retrieval device (Concentric Medical, Mountain View, Calif.).

This invention can be embodied in other forms without departing from the spirit or essential attributes thereof and, accordingly, reference should be had to the following claims rather than the foregoing specification as indicating the scope of the invention.

I claim:

1. An embolectomy device, comprising:
a catheter; and
an elongated shaft positioned in and moveable within the catheter, said shaft having proximal and distal end portions, said shaft having a retrieval portion at or near said distal end portion, said retrieval portion having a plurality of legs having proximal and distal ends and a mid-portion between said proximal and distal ends, said mid-portion of said legs being movable laterally outward from a long axis of said elongated shaft when said retrieval portion has been moved out of said catheter.

2. The embolectomy device of claim 1, further comprising a retrieval net fixed to said legs between said mid-portion and said distal end of said legs.

3. The embolectomy device of claim 1, wherein said elongated shaft is a guide wire.

4. The embolectomy device of claim 2, wherein said legs are spring arms.

5. The embolectomy device of claim 4, wherein said spring arms comprise a bend at said mid portion defining proximal and distal portions, said bend biasing said spring arms to a lateral outward position.

6. The embolectomy device of claim 2, wherein said net is constructed from a polymeric material.

7. The embolectomy device of claim 1, wherein said legs are integral with said elongated shaft.

8. The embolectomy device of claim 1, wherein said legs are affixed to said elongated shaft.

9. The embolectomy device of claim 1, wherein said elongated shaft comprises a distal penetrating portion.

10. An embolectomy device, comprising:
a catheter;
an elongated shaft positioned in and moveable within the catheter, said shaft having proximal and distal end portions, said shaft having a retrieval portion at or near said distal end portion, said retrieval portion having a plurality of legs having proximal and distal portions, distal portions of said legs being movable laterally outward from a long axis of said elongated shaft when said retrieval portion has been at least partially moved out of said catheter; and
a retrieval net fixed to said distal portions of said plurality of legs.

11. An embolectomy device, comprising:
an outer catheter; and
an elongated shaft positioned in and moveable within said outer catheter, said elongated shaft having connected thereto an expander, whereby when said expander is moved from within said outer catheter said expander can expand to engage a clot.

12. The embolectomy device of claim 11, wherein said elongated shaft comprises an inner catheter, said expander being connected to said inner catheter.

13. The embolectomy device of claim 12, wherein within said inner catheter is an elongated retrieval shaft, said retrieval shaft having proximal and distal end portions, said retrieval shaft having a retrieval portion at or near said distal end portion, said retrieval portion having a plurality of legs having proximal and distal ends and a mid-portion between said proximal and distal ends, said mid-portion of said legs being movable laterally outward from a long axis of said elongated shaft when said retrieval portion has been moved out of said inner catheter.

14. The embolectomy device of claim 12, wherein within said inner catheter is an elongated retrieval shaft positioned in and moveable within the catheter, said retrieval shaft having proximal and distal end portions and a retrieval portion at or near said distal end portion, said retrieval portion having a plurality of legs having proximal and distal portions, distal portions of said legs being movable laterally outward from a long axis of said elongated retrieval shaft when said retrieval portion has been at least partially moved out of said inner catheter; and a retrieval net fixed to said distal portions of said plurality of legs.

15. The embolectomy device of claim 11, wherein said expander is at least partially covered by a net.

16. The embolectomey device of claim 15, wherein said net comprises a biocompatible or non-thrombogenic material.

17. A method of performing an embolectomy, comprising the steps of:
providing an embolectomy device comprising an outer catheter; an elongated shaft positioned in and moveable within said outer catheter, said elongated shaft having connected thereto an expandable expander, whereby when said expander is moved from within said outer catheter said expander will expand to engage an occlusion;
manipulating a distal portion of said outer catheter to a location adjacent to an occlusion in a body canal;
moving at least the expander into the occlusion; and
expanding the expander to move the occlusion toward said body canal, thereby creating a lumen.

18. The method of claim 17, wherein said elongated shaft comprises an inner catheter, said expander being connected to said inner catheter.

19. The method of claim 18, wherein within said inner catheter is an elongated retrieval shaft, said retrieval shaft having proximal and distal end portions, said retrieval shaft having a retrieval portion at or near said distal end portion, said retrieval portion having a plurality of legs having proximal and distal ends, a portion of said legs being movable laterally outward from a long axis of said elongated shaft when said retrieval portion has been at least partially moved out of said catheter, and a retrieval net fixed to said movable portions of said plurality of legs;
said method further comprising the step of, after said occlusion has been moved by said expander, moving said retrieval portion out of said inner catheter and moving laterally outward said legs and said net, and then moving said net proximally to remove at least portions of said occlusion from said body canal.

20. The method of claim 18, wherein within said inner catheter is an elongated retrieval shaft, said retrieval shaft having proximal and distal end portions, said method further comprising the step of moving said retrieval portion at least partially out of the catheter, and moving at least a portion of the retrieval portion laterally outward;
moving the retrieval portion proximally so as to engage said occlusion with the retrieval portion; and
removing said microcatheter, elongated shaft, retrieval portion, and portions of said occlusion from said body canal.

21. The embolectomy device of claim 17, wherein said expander is at least partially covered by a net.

22. The embolectomey device of claim 21, wherein said net comprises a biocompatible or non-thrombogenic material.

23. A method of performing an embolectomy, comprising the steps of:
providing an embolectomy device comprising a catheter; an elongated shaft positioned in and moveable within the catheter, said shaft having proximal and distal end portions, said shaft having a retrieval portion at or near said distal end portion, said retrieval portion having a plurality of legs having proximal and distal end portions, distal portions of said legs being movable laterally outward from a long axis of said elongated shaft when said retrieval portion has been moved at least partially out of said catheter, and a retrieval net fixed to said distal portions of said plurality of legs;
manipulating the embolectomy device to a location adjacent to an occlusion in a body canal;
moving at least the retrieval portion through the occlusion;
moving the retrieval portion at least partially out of the catheter, and moving at least a portion of the legs laterally outward;
moving the retrieval portion proximally so as to engage said occlusion with the retrieval net; and
removing said microcatheter, elongated shaft, retrieval portion, and portions of siad occlusion from said body canal.

24. The method of claim 23, wherein said retrieval portion has a plurality of legs having proximal and distal ends and a mid-portion between said proximal and distal ends, said mid-portion of said legs being movable laterally outward from a long axis of said elongated shaft when said retrieval portion has been moved out of said catheter, said method further comprising the step of moving said mid-portion laterally outward when said retrieval portion has been moved out of said catheter.
